SEQUENCE LISTING (28 sequences)

SEQ ID NO: 1 - RNA, 23 nt, Homo sapiens
aaaagugcuuacagugcagguag

SEQ ID NO: 2 - RNA, 21 nt, Homo sapiens
uaaagugcugacagugcagau

SEQ ID NO: 3 - RNA, 22 nt, Homo sapiens
ucguaccgugaguaauaaugcg

SEQ ID NO: 4 - RNA, 23 nt, Homo sapiens
ucuacagugcacgugucuccagu

SEQ ID NO: 5 - RNA, 23 nt, Homo sapiens
uguaguguuuccuacuuuaugga

SEQ ID NO: 6 - RNA, 22 nt, Homo sapiens
ugagaacugaauuccauggguu

SEQ ID NO: 7 - RNA, 22 nt, Homo sapiens
ucucccaacccuuguaccagug

SEQ ID NO: 8 - RNA, 22 nt, Homo sapiens
uagcagcacguaaauauuggcg

SEQ ID NO: 9 - RNA, 23 nt, Homo sapiens
caaagugcuuacagugcagguag

SEQ ID NO: 10 - RNA, 23 nt, Homo sapiens
caacggaaucccaaaagcagcug

SEQ ID NO: 11 - RNA, 22 nt, Homo sapiens
ugggucuuugcgggcgagauga

SEQ ID NO: 12 - RNA, 23 nt, Homo sapiens
ugugcaaauccaugcaaaacuga

SEQ ID NO: 13 - RNA, 23 nt, Homo sapiens
uaaagugcuuauagugcagguag

SEQ ID NO: 14 - RNA, 22 nt, Homo sapiens
ugucaguuugucaaauacccca

SEQ ID NO: 15 - RNA, 22 nt, Homo sapiens
uggcucaguucagcaggaacag

SEQ ID NO: 16 - RNA, 22 nt, Homo sapiens
uguaaacauccucgacuggaag

SEQ ID NO: 17 - RNA, 22 nt, Homo sapiens
uguaaacauccuacacucagcu

SEQ ID NO: 18 - RNA, 23 nt, Homo sapiens
uguaaacauccuacacucucagc

SEQ ID NO: 19 - RNA, 22 nt, Homo sapiens
aaaagcuggguugagagggcga

SEQ ID NO: 20 - RNA, 23 nt, Homo sapiens
gggggggcaggaggggcucaggg

SEQ ID NO: 21 - RNA, 22 nt, Homo sapiens
ucaggcucaguccccucccgau

SEQ ID NO: 22 - RNA, 22 nt, Homo sapiens
uccuguacugagcugccccgag

SEQ ID NO: 23 - RNA, 22 nt, Homo sapiens
gacuauagaacuuucccccuca

SEQ ID NO: 24 - RNA, 22 nt, Homo sapiens
uacccauugcauaucggaguug

SEQ ID NO: 25 - RNA, 22 nt, Homo sapiens
uauugcacuugucccggccugu

SEQ ID NO: 26 - RNA, 19 nt, Homo sapiens
uggauuuuuggaucaggga

SEQ ID NO: 27 - RNA, 22 nt, Homo sapiens
uggagugugacaaugguguuug

SEQ ID NO: 28 - RNA, 22 nt, Homo sapiens
aagacgggaggaaagaagggag

(19) United States
(12) Patent Application Publication
(10) Pub. No.: US 2016/0312301 A1
(43) Pub. Date: Oct. 27, 2016

(54) MICRORNA BIOMARKERS FOR OVARIAN CANCER

(71) Applicant: The Feinstein Institute for Medical Research, Manhasset, NY (US)

(72) Inventors: Annette LEE, Manhasset, NY (US); luliana SHAPIRA, Manhasset, NY (US)

(21) Appl. No.: 15/105,763

(22) PCT Filed: Dec. 22, 2014
(86) PCT No.: PCT/US2014/071840

§ 371 Date: Jun. 17, 2016

Related U.S. Application Data
(60) Provisional application No. 61/919,009, filed on Dec. 20, 2013.

(51) Int. Cl. C12Q 1/68; A61K 33/24

(52) U.S. Cl. CPC C12Q 1/6886; A61K 33/24; C12Q 2600/118; C12Q 2600/158; C12Q 2600/178; C12Q 2600/16

(57) ABSTRACT

A method of diagnosing ovarian cancer in a subject is described that includes identifying the expression level of at least one diagnostic miRNA in a biological sample from the subject, comparing the expression level of the at least one diagnostic miRNA to control expression levels, and diagnosing the subject as having or being at an increased risk of having ovarian cancer if the subject has a changed expression level of the one or more diagnostic miRNA. The method also includes a method of providing a prognosis for a subject having ovarian cancer, and kits for carrying out a diagnosis or prognosis of ovarian cancer using miRNA.

### CROSS-REFERENCE TO RELATED APPLICATIONS

This application claims priority to U.S. Provisional Application Ser. No. 61/919,009, filed on Dec. 20, 2013, which is hereby incorporated by reference in its entirety.

### SEQUENCE LISTING

The instant application contains a Sequence Listing which has been submitted in ASCII format via EFS-Web and is hereby incorporated by reference in its entirety. Said ASCII copy, created on Dec. 17, 2014, is named Ovarian Cancer Sequences NSLIJ-023009 WO ORD_ST25 and is 4,280 bytes in size.

### BACKGROUND

Each year, the American Cancer Society estimates over 20,000 new cases of ovarian cancer will be diagnosed and approximately 15,500 women die of the disease. Although 6% of all cancer death in women is caused by ovarian cancer, there has been relatively little improvement in survival rates over the past decade. Five year survival is critically dependent on the ovarian cancer stage at diagnosis; if diagnosed and treated while localized (stage I, II), 5 year survival rates can reach over 90%. However, most ovarian cancer cases are diagnosed as advanced disease (stage III, IV) where 5 year survival is less than 30%.

Disease outcome is significantly higher with early diagnosis, however, currently there is no noninvasive method to accurately detect early stage ovarian cancer. Anderson et al., J Natl Cancer Inst., 102(1):26-38 (2010). This was recently re-confirmed by the US Preventative Services Task Force that annual screenings with transvaginal ultrasound and measurement of the cancer antigen, CA-125 serum levels does not significantly increase survival rates in asymptomatic women with no genetic risk mutations. In fact, they concluded that screening for ovarian cancer can lead to unnecessary death from surgical complications associated with a false positive result. Moyer, V A, Ann Intern Med 157(12): p. 900-904 (2012). Other biomarkers such as mesothelin and human epididymis 4 also have limited diagnostic utility. There clearly exists an unmet need for a noninvasive diagnostic tool with sufficient sensitivity and specificity to positively impact survival rates in ovarian cancer.

MiRNAs are small (15-30 nt), noncoding RNAs that regulate gene expression post-transcriptionally by binding in the 3′ untranslated region (3′UTR) of their specific messenger RNAs and interfering with translation. The presence and expression levels of specific tissue miRNAs have been associated with different stages in ovarian cancer and clinical outcome. Iorio et al. were the first to compare genome-wide miRNA expression profiles from both ovarian cancer and normal ovary tissue. Iorio et al., Cancer Res., 67, 8699-8707 (2007). Approximately 30 miRNAs were found to be differentially expressed between normal and cancerous tissue. Since then several others have confirmed the dysregulation of miRNAs in ovarian cancer tissue. Creighton et al., PLoS One 7(3):e34546 (2012); Dahiya N, Morin P J, Endocr Relat Cancer, 17(1):F77-F89 (2010).

More recently, it has become apparent that miRNAs circulate in the peripheral blood in several compartments. They may be found within exosomes in the plasma or free of cellular material and bound to proteins such as Argonaut 2. Arroyo et al., Proc Natl Acad Sci USA, 108(12):5003-5008 (2011). Thus they offer a potential biomarker for a wide range of diseases. Devaux et al., Clin Chem. 58(3):559-567 (2012); Shen et al., Cancer Lett., 329(2):125-136 (2013). Indeed, there are several reports of circulating miRNAs associated with ovarian cancer and correlated with detection, severity of disease and response to treatment, although the methods for sample preparation and miRNA detection vary between studies. Resnick et al., Gynecol Oncol 112(1):55-59 (2009); Taylor D D, Gercel-Taylor C., Gynecol Oncol 110(1):13-21 (2008); Häusler et al., Br J Cancer, 103(5):693-700 (2010).

### SUMMARY OF THE INVENTION

Securing a diagnosis of ovarian cancer and establishing means to predict outcomes to therapeutics remain formidable clinical challenges. Early diagnosis is particularly important since survival rates are markedly improved if tumor is detected early.

Comprehensive miRNA profiles were generated on pre-surgical plasma samples from 42 women with confirmed serous epithelial ovarian cancer, 36 women diagnosed with a benign neoplasm, and 23 comparably age matched women with no known pelvic mass.

Twenty-two miRNAs were differentially expressed between healthy controls and the ovarian cancer group (p<0.05), while a six miRNA profile subset distinguished pre-surgical plasma from benign and ovarian cancer patients. There were also significant differences in miRNA profiles in pre-surgical plasma from women diagnosed with ovarian cancer who had short overall survival when compared to women with long overall survival (p<0.05).

The preliminary data supports the utility of circulating plasma miRNAs to distinguish women with ovarian cancer from those with a benign mass and identify women likely to benefit from currently available treatment for serous epithelial ovarian cancer from those who may not.

### BRIEF DESCRIPTION OF THE FIGURES

The present invention may be more readily understood by reference to the following figures, wherein:

FIG. 1 provides a graph showing the level of miR-1290 in presurgical plasma samples. Individual levels of miR-1290 detected in plasma samples from subjects with ovarian cancer, benign mass or healthy controls. UND—undeclared survival, n=16; LOS—long overall survival n=7; SOS—short overall survival n=19; CNTRL—healthy control, n=23; BGN—benign mass, n=32) Expressed as—(ΔCt).

FIG. 2 provides graphs providing a comparison of miRNA plasma levels: pre-surgical vs post-chemotherapy. The MiRNAs demonstrate significant differences between pre-surgical and post-chemotherapy.

FIG. 3 provides a bar graph and list of values comparing the fold change in plasma miRNA levels in long overall survival subjects presurgery versus post chemotherapy, based on plasma samples of women with long overall survival (n=5). Expressed as—(ΔCt).

### DETAILED DESCRIPTION OF THE INVENTION

The present invention relates methods of providing a diagnosis for a subject who may have ovarian cancer, or a prognosis for a subject having been diagnosed with ovarian cancer. The diagnosis or prognosis is provided by identifying the presence of particular microRNA which the inventors have associated with ovarian cancer, and in some cases particularly risky forms of ovarian cancer. The method is particularly useful for distinguishing between a benign growth and actual ovarian cancer, without the need for obtaining a tissue biopsy.

### DEFINITIONS

As used herein, the term “diagnosis” can encompass determining the likelihood that a subject will develop a disease, or the existence or nature of disease in a subject. The term diagnosis, as used herein also encompasses determining the severity and probable outcome of disease or episode of disease or prospect of recovery, which is generally referred to as prognosis).

As used herein, the term “prognosis” refers to a prediction of the probable course and outcome of a disease, or the likelihood of recovery from a disease. Prognosis is distinguished from diagnosis in that it is generally already known that the subject has the disease, although prognosis and diagnosis can be carried out simultaneously. In the case of a prognosis for ovarian cancer, the prognosis categorizes the relative severity of the ovarian cancer, and in particular the risk of metastasis, which can be used to guide selection of appropriate therapy for the ovarian cancer.

As used herein, the terms “treatment,” “treating,” and the like, refer to obtaining a desired pharmacologic or physiologic effect. The effect may be therapeutic in terms of a partial or complete cure for a disease or an adverse effect attributable to the disease. “Treatment,” as used herein, covers any treatment of a disease in a mammal, particularly in a human, and can include inhibiting the disease or condition, i.e., arresting its development; and relieving the disease, i.e., causing regression of the disease.

The term therapy, as used herein, encompasses activity carried out to treat a disease. The specific activity carried out to conduct therapy can include use of surgery, radiotherapy, hormonal therapy, chemotherapy, or the use of one or more therapeutic agents (e.g., anticancer agents).

The terms “therapeutically effective” and “pharmacologically effective” are intended to qualify the amount of an agent which will achieve the goal of improvement in disease severity and the frequency of incidence over treatment of each agent by itself, while avoiding adverse side effects typically associated with alternative therapies. The effectiveness of treatment may be measured by evaluating a reduction in tumor load or decrease in tumor growth in a subject in response to the administration of anticancer agents. The reduction in tumor load may be represent a direct decrease in mass, or it may be measured in terms of tumor growth delay, which is calculated by subtracting the average time for control tumors to grow over to a certain volume from the time required for treated tumors to grow to the same volume.

As used herein, the term “expression level,” particularly as applied to microRNA, refers to the absolute amount or relative amount of the microRNA in the sample. According to certain embodiments of the present disclosure, the term “expression level” means the normalized level of the microRNA. Expression levels may be normalized with respect to the expression level of one or more reference (housekeeping) microRNAs (e.g., an internal control microRNA), or the use of average expression levels from healthy subjects, which are referred to herein as control expression levels. Persons having ordinary skills in the art would recognize that numerous methods of normalization are known, and could be applied for use in the methods described herein. “Differential expression,” as used herein, refers to quantitative differences in the expression of microRNA in comparison to corresponding controls. The degree of a decrease or increase in miR expression can be any percentage value. For example, it can be 25% or more, 50% or more, or 75% or more as a percentage relative to a control, or corresponding reductions in expression.

“Expression profile” as used herein may mean a genomic expression profile, e.g., an expression profile of microRNAs. Profiles may be generated by any convenient means for determining a level of a nucleic acid sequence e.g. quantitative hybridization of microRNA, labeled microRNA, amplified microRNA, cRNA, etc., quantitative PCR, ELISA for quantitation, and the like, and allow the analysis of differential gene expression between two samples. A subject or patient tumor sample, e.g., cells or collections thereof, e.g., tissues, is assayed. Nucleic acid sequences of interest are nucleic acid sequences that are found to be predictive, including the nucleic acid sequences provided above, where the expression profile may include expression data for 5, 10, 20, 25, or more of, including all of the microRNA described herein. The term “expression profile” may also mean measuring the abundance of the nucleic acid sequences in the measured samples.

“Nucleic acid” or “oligonucleotide” or “polynucleotide”, as used herein, may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single-stranded or double-stranded, or may contain portions of both double-stranded and single-stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

The term “microRNAs” as used herein refers to a class of small RNAs typically between 15 and 30 nucleotides long. microRNAs can refer to a class of small RNAs that play a role in gene regulation. In a preferred embodiment, a microRNA refers to a human, small RNA of 20, 21, 22, 23, 24, 25, or 26 nucleotides long. MicroRNA can also be referred to as miR or miRNA. See Bartel D P, Cell, 136(2):215-233 (2009).

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” refers to +/−10% deviation from the basic value.

Unless defined otherwise, all technical and scientific terms used herein have the same meaning as commonly understood by one of ordinary skill in the art to which this invention belongs.

As used herein and in the appended claims, the singular forms “a”, “and”, and “the” include plural referents unless the context clearly dictates otherwise. Thus, for example, reference to “a sample” also includes a plurality of such samples and reference to “a microRNA” includes reference to one or more microRNA molecules, and so forth.

### Methods of Diagnosing Ovarian Cancer

One aspect of the invention provides a method of diagnosing ovarian cancer in a subject. The method includes identifying the expression level of at least one diagnostic miRNA selected from the group consisting of miR-106a, miR-106b, miR-126-3p, miR-1274a, miR-139-5p, miR-142-3p, miR-146a, miR-150, miR-16, miR-17, miR-191, miR-193a-5p, miR-30a-5p, miR-19b, miR-20a, miR-223, miR-24, miR-30b, miR-30c, miR-320, miR-328, miR-484, miR-486, miR-625-3p, miR-660, miR-720, miR-92a, miR-122-5p, miR-483-5p, and miR-127-4b in a biological sample from the subject, comparing the expression level of the at least one diagnostic miRNA to control expression levels, and diagnosing the subject as having or being at an increased risk of having ovarian cancer if the subject has a changed expression level of the one or more diagnostic miRNA. A diagnostic miRNA is a miRNA whose expression changes as a result of the development of ovarian cancer.

The sequences of diagnostic and/or prognostic miRNA that can be used for the methods described herein are provided in Table 1. With regard to miRNA whose sequences are not specifically described herein, it should be noted that the specific nucleotide sequence of particular miRs are known to those skilled in the art, and can readily be obtained through reference to the miRBase database of Cambridge University. Griffiths-Jones et al., Nucleic Acids Research, 34, D140-D144 (2006), the disclosure of which is incorporated herein by reference. It should also be noted that the nomenclature used for miRNA has been evolving, and that closely related names, such as miR-126 and miR-126-3p, can refer to the same miR.

TABLE 1
Sequences of MicroRNA associated with Ovarian Cancer

| Designation | Nucleotide Sequence | Sequence ID |
|---|---|---|
| hsa-miR-106a-5p | AAAAGUGCUUACAGUGCAGGUAG | SEQ ID NO: 1 |
| hsa-miR-106b-5p | UAAAGUGCUGACAGUGCAGAU | SEQ ID NO: 2 |
| hsa-miR-126-3p | UCGUACCGUGAGUAAUAAUGCG | SEQ ID NO: 3 |
| hsa-miR-139-5p | UCUACAGUGCACGUGUCUCCAGU | SEQ ID NO: 4 |
| hsa-miR-142-3p | UGUAGUGUUUCCUACUUUAUGGA | SEQ ID NO: 5 |
| hsa-miR-146a-5p | UGAGAACUGAAUUCCAUGGGUU | SEQ ID NO: 6 |
| hsa-miR-150-5p | UCUCCCAACCCUUGUACCAGUG | SEQ ID NO: 7 |
| hsa-miR-16-5p | UAGCAGCACGUAAAUAUUGGCG | SEQ ID NO: 8 |
| hsa-miR-17-5p | CAAAGUGCUUACAGUGCAGGUAG | SEQ ID NO: 9 |
| hsa-miR-191-5p | CAACGGAAUCCCAAAAGCAGCUG | SEQ ID NO: 10 |
| hsa-miR-193a-5p | UGGGUCUUUGCGGGCGAGAUGA | SEQ ID NO: 11 |
| hsa-miR-19b-3p | UGUGCAAAUCCAUGCAAAACUGA | SEQ ID NO: 12 |
| hsa-miR-20a-5p | UAAAGUGCUUAUAGUGCAGGUAG | SEQ ID NO: 13 |
| hsa-miR-223-3p | UGUCAGUUUGUCAAAUACCCCA | SEQ ID NO: 14 |
| hsa-miR-24-3p | UGGCUCAGUUCAGCAGGAACAG | SEQ ID NO: 15 |
| hsa-miR-30a-5p | UGUAAACAUCCUCGACUGGAAG | SEQ ID NO: 16 |
| hsa-miR-30b-5p | UGUAAACAUCCUACACUCAGCU | SEQ ID NO: 17 |
| hsa-miR-30c-5p | UGUAAACAUCCUACACUCUCAGC | SEQ ID NO: 18 |
| hsa-miR-320a | AAAAGCUGGGUUGAGAGGGCGA | SEQ ID NO: 19 |
| hsa-miR-328-5p | GGGGGGGCAGGAGGGGCUCAGGG | SEQ ID NO: 20 |
| hsa-miR-484 | UCAGGCUCAGUCCCCUCCCGAU | SEQ ID NO: 21 |
| hsa-miR-486-5p | UCCUGUACUGAGCUGCCCCGAG | SEQ ID NO: 22 |
| hsa-miR-625-3p | GACUAUAGAACUUUCCCCCUCA | SEQ ID NO: 23 |
| hsa-miR-660-5p | UACCCAUUGCAUAUCGGAGUUG | SEQ ID NO: 24 |
| hsa-miR-92a-3p | UAUUGCACUUGUCCCGGCCUGU | SEQ ID NO: 25 |
| hsa-miR-1290 | UGGAUUUUUGGAUCAGGGA | SEQ ID NO: 26 |
| hsa-miR-122-5p | UGGAGUGUGACAAUGGUGUUUG | SEQ ID NO: 27 |
| hsa-miR-483-5p | AAGACGGGAGGAAAGAAGGGAG | SEQ ID NO: 28 |

In some embodiments, the changed level is an increased level, and the diagnostic miRNA is selected from the group consisting of miR-1274a, miR-625-3p, miR-720, miR-483-5p, and miR-1274b. In other embodiments, the changed level is a decreased level, and wherein the diagnostic miRNA is selected from the group consisting of miR-106a, miR-106b, miR-126-3p, miR-139-5p, miR-142-3p, miR-146a, miR-150, miR-16, miR-17, miR-191, miR-193a-5p, miR-19b, miR-20a, miR-223, miR-24, miR-30b, miR-30c, miR-320, miR-328, miR-484, miR-486, miR-660, miR-92a, and miR-122-5p. In further embodiments, diagnostic miRNA that have a high change in expression level can be used. For example, in some embodiments, the diagnostic miRNA is selected from the group consisting of miR-106a, miR-126-3p, miR-146a, miR-150, miR-16, miR-17, miR-19b, miR-20a, miR-223, miR-24, and miR-92a.

In other embodiments, the expression level of a plurality of diagnostic miRNAs are identified when carrying out the method. For example, the method can involve evaluating the expression level of 2, 3, 4, 5, 6, 7, 10, 11, 12, 13, 14, 15, 16, 17, 18, 19, or 20 or more miRNA. When a plurality of diagnostic miRNA are identified, it may be preferable to identify the expression levels of the miRNA using a microarray.

### Methods of Providing a Prognosis for a Subject Having Ovarian Cancer

Another aspect of the invention provides a method of providing a prognosis for a subject having ovarian cancer. The method includes identifying the expression level of at least one prognostic miRNA selected from the group consisting of miR-720, miR20a, miR-223, miR-126-3p, and miR-1290 in a biological sample from the subject, comparing the expression level of the at least one prognostic miRNA to control expression levels, and identifying the subject as having a poor prognosis if the subject has a changed level of the one or more prognostic miRNA. A prognostic miRNA is a miRNA whose expression changes in relation to the severity of the ovarian cancer.

In some embodiments, the changed level is an increased level, and the prognostic miRNA is selected from miR-720 and/or miR-20a. In other embodiments, the changed level is a decreased level, and wherein the prognostic miRNA is selected from the group consisting of miR-223, miR-126-3p, and miR-1290. In further embodiments, diagnostic miRNA that have a high change in expression level can be used. For example, in some embodiments, the prognostic miRNA is miR-1290.

In other embodiments, the expression level of a plurality of prognostic miRNAs are identified when carrying out the method. For example, the method can involve evaluating the expression level of 2, 3, 4, 5, 6, 7, 10, 11, 12, 13, 14, 15, 16, 17, 18, 19, or 20 or more miRNA. When a plurality of prognostic miRNA are identified, it may be preferable to identify the expression levels of the miRNA using a microarray.

### Ovarian Cancer

As used herein, the terms “tumor” or “cancer” refer to a condition characterized by anomalous rapid proliferation of abnormal cells of a subject. The abnormal cells often are referred to as “neoplastic cells,” which are transformed cells that can form a solid tumor. The term “tumor” refers to an abnormal mass or population of cells (e.g., two or more cells) that result from excessive or abnormal cell division, whether malignant or benign, and precancerous and cancerous cells. Malignant tumors are distinguished from benign growths or tumors in that, in addition to uncontrolled cellular proliferation, they can invade surrounding tissues and can metastasize, which refers to the spread of the cancer to other tissue sites.

The present invention provides methods for the diagnosis and prognosis of subjects with ovarian cancer, which is a cancer that begins in an ovary. The most common type of ovarian cancer, comprising more than 95% of cases, is ovarian carcinoma. Ovarian cancers are histologically and genetically divided into two types, Type I and Type II. Type I cancers are of low histological grade, and include endometrioid, mucinous, and clear cell carcinomas. Type II cancers are of higher histological grade and include serous carcinoma and carcinosarcoma. In addition, there are several subtypes of ovarian carcinoma. These include surface epithelial stromal tumor (ovarian epithelial carcinoma), papillary serous cystadenocarcinoma, adenocarcinoma, endometrioid tumor, serious cystadenocarcinoma, and papillary ovarian cancer. In some embodiments, the ovarian cancer is serous epithelial ovarian cancer.

In some embodiments, the subject has been diagnosed with one or more symptoms of ovarian cancer. Diagnosis of ovarian cancer can include a physical examination (including a pelvic examination), a blood test (for CA-125 or other markers associated with ovarian cancer), or a transvaginal ultrasound. Inspection of the abdominal cavity, obtaining and analyzing tissue biopsies, and looking for cancer cells in the abdominal fluid can also be used to diagnose ovarian cancer. Symptoms of ovarian cancer include bloating, pelvic pain, and abdominal swelling.

### Biological Samples

“Biological sample” as used herein means a sample of biological tissue or fluid that comprises nucleic acids. Such samples include, but are not limited to, tissue or fluid isolated from subjects. Biological samples may also include sections of tissues such as biopsy and autopsy samples, FFPE samples, frozen sections taken for histological purposes, blood, plasma, serum, sputum, stool, tears, mucus, urine, or vaginal secretions. Biological samples also include explants and primary and/or transformed cell cultures derived from animal or patient tissues. A biological sample may be provided by removing a sample of cells from an animal, but can also be accomplished by using previously isolated cells (e.g., isolated by another person, at another time, and/or for another purpose), or by performing the methods described herein in vivo.

The level of the target miR gene product is measured in a biological sample obtained from a subject. For example, a biological sample can be removed from a subject suspected of having ovarian cancer. Such a biological sample can include a tissue or cell biopsy obtained from a region of the ovaries suspected to be precancerous or cancerous. Alternatively, a biological sample can include blood, serum, or plasma obtained from the subject. In some embodiments, the biological sample is plasma, such as plasma that has been ultracentrifuged before determining the level of diagnostic miRNA in the plasma.

The methods involve providing or obtaining a biological sample from the subject, which can be obtained by any known means including needle stick, needle biopsy, swab, and the like. In an exemplary method, the biological sample is a blood sample, which may be obtained for example by venipuncture.

A biological sample may be fresh or stored. Biological samples may be or have been stored or banked under suitable tissue storage conditions. The biological sample may be a tissue sample expressly obtained for the assays of this invention or a tissue sample obtained for another purpose which can be subsampled for the assays of this invention. Preferably, biological samples are either chilled or frozen shortly after collection if they are being stored to prevent deterioration of the sample. Biological samples may also be stored in RNAlater™ for analysis at a later date.

The sample may be pretreated as necessary by dilution in an appropriate buffer solution, heparinized, concentrated if desired, or fractionated by any number of methods including but not limited to ultracentrifugation, fractionation by fast performance liquid chromatography (FPLC) or HPLC, or precipitation of proteins with dextran sulfate or other methods. For example, in some embodiments, the biological sample is plasma that is ultracentrifuged before determining the level of diagnostic or prognostic miRNA in the plasma. Any of a number of standard aqueous buffer solutions at physiological pH, such as phosphate, Tris, or the like, can be used.

### Subjects

The terms “individual,” “subject,” and “patient” are used interchangeably herein irrespective of whether the subject has or is currently undergoing any form of treatment. As used herein, the term “subject” generally refers to any vertebrate, including, but not limited to a mammal. Examples of mammals including primates, including simians and humans, equines (e.g., horses), canines (e.g., dogs), felines, various domesticated livestock (e.g., ungulates, such as swine, pigs, goats, sheep, and the like), as well as domesticated pets (e.g., cats, hamsters, mice, and guinea pigs). Diagnosis or prognosis of humans is of particular interest.

In some embodiments, the subject is a subject identified as being at increased risk of having, or having a more severe form, of ovarian cancer than a typical subject. A more severe form of cancer can include cancers at a more advanced stage, or cancer's having an increased risk of developing metastasis. For example, women who ovulate more have an increased risk of having ovarian cancer. Thus, those who have never had children are at increased risk, as are those who begin ovulation at a younger age or reach menopause at an older age. Other risk factors include hormone therapy after menopause, fertility medication and obesity. Ovarian cancer is associated with increased age and family history of ovarian cancer, which is associated with a 9.8-fold higher risk. About 10% of cases are related to inherited genetic risk, and those with the gene mutations BRCA1 or BRCA2 have approximately a 50% chance of developing the disease. The most common gene mutations in ovarian cancer occur in NF1, BRCA1, BRCA2, and CDK12, which are therefore also associated with increased risk. Type I ovarian cancers tend to have microsatellite instability in several genes, including BRAF, KRAS, and PTEN, which are tumor suppressor genes, while type II cancers have different genes mutated, including p53, BRCA1, and BRCA2. In 50% of high-grade serous cancers, homologous recombination DNA repair is dysfunctional, as are the Notch and FOXM1 signaling pathways. Alternately, subjects can have a decreased risk of having ovarian cancer if they have used hormonal birth control, had a tubal ligation, or have provided breast feeding.

A subject at increased risk is more likely to suffer greater symptoms, including death, if not provided with effective treatment, as compared with a subject who is not at increased risk. In some embodiments, the level of increased risk can be characterized as a percentage increase relative to the risk of a typical subject having ovarian cancer. For example, a subject at increased risk can be at a 10%, 20%, 30%, 40%, 50%, 60%, 70%, 80%, 90%, 100%, or greater than 100% level of increased risk as compared with a typical subject.

Methods for Detecting microRNA

The expression level of the microRNA may be determined by any method known in the art, examples of which include but are not limited to amplification-based methods such as polymerase chain reaction (PCR), quantitative RT-PCR (qPCR), real-time quantitative PCR (RT-qPCR), semi-quantitative RT-PCR, ligase chain reaction (LCR), quantitative nuclease protection assay (qNPA), in situ hybridization, and strand displacement amplification (SDA).

In some embodiments, the microRNA is detected by hybridization with another nucleic acid sequence. The type of the nucleotide of the nucleic acid sequence is not particularly limited provided that it can specifically hybridize to the microRNA of the present invention. The length of the part of the polynucleotide is not particularly limited provided that it specifically hybridizes to the predetermined microRNA according to the present invention; however, it is preferably 10 to 100 mers, more preferably 10 to 40 mers in view of securing the stability of hybridization. The polynucleotide or a part thereof can be obtained by chemical synthesis or the like using a method well known in the art.

In some embodiments, the microRNA is detected by quantitative polymerase chain reaction. The quantitative PCR method is not particularly limited provided that it is a method using a primer set capable of amplifying the sequence of the microRNA and can measure the expression level of the present microRNA; conventional quantitative PCR methods such as an agarose electrophoresis method, an SYBR green method, and a fluorescent probe method may be used. However, the fluorescent probe method is most preferable in terms of the accuracy and reliability of quantitative determination.

The primer set for the quantitative PCR method means a combination of primers (polynucleotides) capable of amplifying the sequence of the microRNA. The primers are not particularly limited provided that they can amplify the sequence of the microRNA; examples thereof can include a primer set consisting of a primer consisting of the sequence of a 5′ portion of the sequence of a microRNA of the present invention (forward primer) and a primer consisting of a sequence complementary to the sequence of a 3′ portion of the microRNA (reverse primer). Here, the 5′ means 5′ to the sequence corresponding to the reverse primer when both primers were positionally compared in the sequence of a mature microRNA; the 3′ means 3′ to the sequence corresponding to the forward primer when both primers were positionally compared in the sequence of a microRNA.

Preferred examples of the 5′ sequence of a microRNA can include a sequence 5′ to the central nucleic acid of the microRNA sequence; preferred examples of the 3′ sequence of the microRNA can include a sequence 3′ to the central nucleic acid of the microRNA sequence. The length of each primer is not particularly limited provided that it enables the amplification of the microRNA; however, each primer is preferably a 7-to-10-mer polynucleotide. The type of the nucleotide of a polynucleotide as the primer is preferably DNA because of its high stability.

A person skilled in the art will appreciate that a number of detection agents can be used to determine the expression of the microRNA. For example, to detect microRNA, probes, primers, complementary polynucleotide sequences or polynucleotide sequences that hybridize to the microRNA can be used. In some embodiments, reverse complementary polynucleotides serve as probes for microRNA. In alternate embodiments, a complementary polynucleotide sequence that hybridizes to the target polynucleotide sequence can be used to detect expression of the microRNA.

In some embodiments, a fluorescent probe is used. The fluorescent probe is not particularly limited provided that it comprises a polynucleotide consisting of a nucleic acid sequence complementary to the sequence of the present microRNA or a part thereof; preferred examples thereof can include a fluorescent probe capable of being used for the TaqMan™ probe method or the cycling probe method; the fluorescent probe capable of being used for the TaqMan™ probe method can be particularly preferably exemplified. Examples of the fluorescent probe capable of being used for the TaqMan™ probe method or the cycling probe method can include a fluorescent probe in which a fluorochrome is labeled 5′ thereof and a quencher is labeled on 3′ thereof. The fluorochrome, quencher, donor dye, acceptor dye used or the like used with a fluorescent probe are commercially available.

In some embodiments, the presence or amount of microRNA is determined using an array or microarray. The microarray method is not particularly limited provided that it can measure the level of the microRNA whose expression changes in response to the presence of ovarian cancer; examples thereof can include a method which involves labeling the RNA extracted from a tissue with a label (preferably a fluorescent label), contacting the RNA with a microarray to which a probe consisting of a polynucleotide (preferably DNA) consisting of a nucleic acid sequence complementary to the microRNA to be identified or a part thereof is fixed for hybridization, washing the microarray, and measuring the expression level of the remaining microRNAs on the microarray. The array to which the polynucleotide or a part thereof is fixed is not particularly limited; however, preferred examples thereof can include a glass substrate and a silicon substrate, and the glass substrate can be preferably exemplified. A method for fixing the polynucleotide or a part thereof to the array is not particularly limited; a well-known method may be used.

In other embodiments, the microarray is a biochip, sometimes referred to as an MMchip in the context of biochips designed for detecting microRNA. The biochip may comprise a solid substrate comprising an attached probe or plurality of probes described herein. The probes may be capable of hybridizing to a target sequence under stringent hybridization conditions. The probes may be attached at spatially defined locations on the substrate. More than one probe per target sequence may be used, with either overlapping probes or probes to different sections of a particular target sequence. The probes may be capable of hybridizing to target miRNA sequences associated with ovarian cancer. The probes may either be synthesized first, with subsequent attachment to the biochip, or may be directly synthesized on the biochip.

### Methods for Treating Ovarian Cancer

In some embodiments, the method of diagnosis or prognosis can be used to indicate or guide treatment of ovarian cancer. For example, the method can further comprise the step of providing suitable treatment if the subject is identified as being at increased risk. Suitable treatment can include use of a variety of different methods of treating cancer, such as surgery, radiation therapy, and administration of hormonal or anticancer agents.

Surgery is often the preferred treatment for ovarian cancer. Surgery involves removal of or more parts of the female reproductive tract, including one (unilateral oophorectomy) or both ovaries (bilateral oophorectomy), the fallopian tubes (salpingectomy), the uterus (hysterectomy), and the omentum (omentectomy). Typically, if a subject is diagnosed with ovarian cancer, all of these are removed. However, for low-grade, unilateral stage IA cancers, only the involved ovary (which must be unruptured) and fallopian tube will be removed.

Another method of treating ovarian cancer is radiation therapy. Some types of ovarian cancer, such as dysgerminomas, can be treated with radiation therapy. However, use of radiation therapy for treatment of ovarian cancer is typically not preferred, because when vital organs are in the radiation field, which is often the case with ovarian cancer, a high dose cannot be safely delivered without serious side effects.

Other methods of treating ovarian cancer include administration of therapeutic agents such as hormonal or anticancer agents. Accordingly, in some embodiments, the method of treatment further comprising the step of administering or prescribing a therapeutic agent targeted to ovarian cancer to a subject diagnosed as having ovarian cancer. Administration of anticancer agents (i.e., chemotherapy) may be used after surgery to treat any residual disease, or may be performed first, followed by surgery. This is called “neoadjuvant chemotherapy,” and is common when a tumor cannot be completely removed or optimally debulked via surgery. If a unilateral salpingo-oophorectomy or other surgery is performed, additional chemotherapy, called “adjuvant chemotherapy” can be given. Chemotherapies used in ovarian cancer include paclitaxel, cisplatin, topotecan, and gemcitabine. Ovarian cancer involving germ cell malignancies are treated differently using a regimen of bleomycin, etoposide, and cisplatin.

### Kits

The present disclosure also provides kits for diagnosing ovarian cancer in a subject. The kits can include one or more detector means specific for at least one diagnostic miRNA selected from the group consisting of miR-106a, miR-106b, miR-126-3p, miR-1274a, miR-139-5p, miR-142-3p, miR-146a, miR-150, miR-16, miR-17, miR-191, miR-193a-5p, miR-19b, miR-20a, miR-223, miR-24, miR-30b, miR-30c, miR-320, miR-328, miR-484, miR-486, miR-625-3p, miR-660, miR-720, miR-92a, miR-122-5p, miR-483-5p, and miR-127-4b, means for comparing the level of the at least one diagnostic miRNA to the level of a corresponding control, and instructions for using the kit to diagnose a subject as having or being at an increased risk of having ovarian cancer when the expression level of the at least one diagnostic miRNA is changed compared to control expression levels.

A kit generally also includes a package with one or more containers holding the reagents, as one or more separate compositions or, optionally, as an admixture where the compatibility of the reagents will allow. The kits may further include enzymes (e.g., polymerases), buffers, labeling agents, nucleotides, controls, and any other materials necessary for carrying out the extraction and/or detection of microRNA. Means for comparing the level of the at least one diagnostic miRNA to the level of a corresponding control can include reference values, reference samples, and items necessary for comparing differing expression levels, such as labeling agents, as known to those skilled in the art. Kits can also include a tool for obtaining a sample from a subject, such as a syringe or a punch tool to obtain a punch-biopsy or needle biopsy.

A kit for diagnosing ovarian cancer in a subject includes one or more detector means specific for at least one diagnostic miRNA. The detector means can include primers or probes designed to detect one or more diagnostic miRNA, and can also include one or more reagents for detecting the miRNA using a PCR-based amplification technology such as RT-PCR or reverse transcription RT-PCR.

In some embodiments, the changed level is an increased level, and the diagnostic miRNA detected by the kit is selected from the group consisting of miR-1274a, miR-625-3p, miR-720, miR-483-5p, and miR-1274b. In other embodiments, the changed level is a decrease level, and the diagnostic miRNA is selected from the group consisting of miR-106a, miR-106b, miR-126, miR-139-5p, miR-142-3p, miR-146a, miR-150, miR-16, miR-17, miR-191, miR-193a-5p, miR-19b, miR-20a, miR-223, miR-24, miR-30b, miR-30c, miR-320, miR-328, miR-484, miR-486, miR-660, miR-92a, and miR-122-5p.

In a further embodiment, the invention provides kits for carrying out a method of providing a prognosis for a subject having ovarian cancer. The kits can include one or more detector means specific for at least one prognostic miRNA selected from the group consisting of miR-720, miR20a, miR-223, miR-126-3p, and miR-1290, with kits for detecting expression levels of miR-1290 being preferred. The kits can also include any of the materials described for use in diagnostic kits, such as instructions, reagents, or packaging.

In some embodiments, the kit includes a plurality of different detector means suitable for determining the expression level of a plurality of diagnostic miRNA. For example, the kit may include different primers or probes for detecting a plurality of different miRNA. In some embodiments, the kit can also include a microarray or biochip for determining the expression level of a plurality of diagnostic miRNA.

“Probe”, as used herein, may mean an oligonucleotide capable of binding to a target nucleic acid of complementary sequence through one or more types of chemical bonds, usually through complementary base pairing, usually through hydrogen bond formation. Probes may bind target sequences lacking complete complementarity with the probe sequence, depending upon the stringency of the hybridization conditions. There may be any number of base pair mismatches which will interfere with hybridization between the target sequence and the single-stranded nucleic acids described herein. However, if the number of mutations is so great that no hybridization can occur under even the least stringent of hybridization conditions, the sequence is not a complementary target sequence. A probe may be single-stranded or partially single- and partially double-stranded. The strandedness of the probe is dictated by the structure, composition, and properties of the target sequence. Probes may be directly labeled or indirectly labeled such as with biotin to which a streptavidin complex may later bind.

The term “complementary” as used herein refers to nucleotide sequences that complement the polynucleotides' reverse sequence. Complementarity is the base principle of DNA replication and transcription as it is a property shared between two DNA or RNA sequences, such that when they are aligned antiparallel to each other, the nucleotide bases at each position in the sequences will be complementary. Complementarity is achieved by distinct interactions between nucleobases: adenine, thymine (uracil in RNA), guanine and cytosine. Adenine (A) and guanine (G) are purines, while thymine (T), cytosine (C) and uracil (U) are pyrimidines. Purines are larger than pyrimidines. Both types of molecules complement each other and can only base pair with the opposing type of nucleobase. In nucleic acid, nucleobases are held together by hydrogen bonding, which only works efficiently between adenine and thymine and between guanine and cytosine. The base complement A=T shares two hydrogen bonds, while the base pair GC has three hydrogen bonds.

The degree of complementarity between two nucleic acid strands may vary, from complete complementarity (each nucleotide is across from its opposite) to no complementary (each nucleotide is not across from its opposite) and determines the stability of the sequences to be together. Lesser degrees of complementarity are referred to herein by percentages of sequence identity as compared with a sequence having 100% complementarity. Embodiments of the invention include sequences having at least about 70% to at least about 100% sequence identify to a complementary sequence. For example, probes can have sequences having at least 70%, 75%, 80%, 85%, 90%, 95%, or 100% sequence identify with a complementary probe. In another example, the sequence identity can be at least about 80% to at least about 95% that of a complementary sequence. In a preferred embodiment, the probe can have at least about 87%, 88%, 89%, 90%, 91%, or 92% sequence identity to a complementary probe. In another preferred embodiment, the probe can have at least 90% sequence identity to a complementary probe.

In some embodiments, a kit comprises one or more pairs of primers (a “forward primer” and a “reverse primer”) for amplification of a cDNA reverse transcribed from a target RNA for carrying out PCR or RT-PCR. Accordingly, in some embodiments, a first primer comprises a region of at least 4, at least 5, at least 6, at least 7, at least 8, at least 9, or at least 10 contiguous nucleotides having a sequence that is identical to the sequence of a region of at least 4, at least 5, at least 6, at least 7, at least 8, at least 9, or at least 10 contiguous nucleotides at the 5′-end of a target RNA. Furthermore, in some embodiments, a second primer comprises a region of at least 4, at least 5, at least 6, at least 7, at least 8, at least 9, or at least 10 contiguous nucleotides having a sequence that is complementary to the sequence of a region of at least 4, at least 5, at least 6, at least 7, at least 8, at least 9, or at least 10 contiguous nucleotides at the 3′-end of a target RNA. In some embodiments, the kit comprises at least a first set of primers for amplification of a cDNA that is reverse transcribed from a target RNA capable of specifically hybridizing to a nucleic acid comprising a sequence identically present in one of SEQ ID NOs: 1 to 28 and/or a cDNA that is reverse transcribed from a target RNA.

The kit can also include instructions for using the kit to carry out a method of diagnosing or providing a prognosis for a subject having ovarian cancer. Instructions included in kits can be affixed to packaging material or can be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

Examples have been included to more clearly describe particular embodiments of the invention. However, there are a wide variety of other embodiments within the scope of the present invention, which should not be limited to the particular example provided herein.

### EXAMPLES

### Example 1

### Circulating Biomarkers for Detection of Ovarian Cancer and Predicting Cancer Outcomes

In this study, using an ultracentrifugation method, the inventors focused on miRNA that is circulating freely in the plasma unbound to cellular material as a potential biomarker for ovarian cancer detection and outcome.

### Materials and Methods

Samples

Peripheral blood samples were collected prior to treatment from women being evaluated for a suspicious pelvic mass. The inventors enrolled 42 women with confirmed serous epithelial ovarian cancer and 36 women diagnosed with a benign neoplasm. Additional blood samples were collected from women diagnosed with ovarian cancer >2 weeks post surgery and >24 months post chemotherapy. Blood samples were also collected from 23 healthy female volunteers. The characteristics of study subjects are given in Table 2.

miRNA Extraction and Profiling

Whole blood, collected in EDTA tubes, was centrifuged at 4K rpm for 10 minutes at room temperature within 5 hours of venipuncture. Plasma was removed, aliquoted and stored at −80° C. One milliliter of thawed plasma was ultracentrifuged at 100,000×g for 60 minutes at 4° C. to remove cell debris, microvessicles and exosomes. Total small RNA from the supernatant was isolated using mirVana PARIS kit (Ambion) according to manufacturer's instruction. MiRNA quality was assessed using Agilent's 2100 bioanalyzer (Agilent Technologies).

Total miRNA profiles were generated using ABI Taqman OpenArray MicroRNA pools A and B to measure the expression of 754 known miRNAs. Briefly total small RNA isolated from 1 ml of purified plasma was divided equally for each sample and used with TaqMan Megaplex RT primer pools A or B to generate cDNA which was subsequently amplified using corresponding Megaplex PreAmp Primers (pools A or B, respectively) following the manufacturer's instructions. Real Time PCR was performed on the Taqman Open Array MicroRNA plates using the Applied Biosystem Open Array Real-Time PCR system. Data were processed using The OpenArray Real-Time qPCR Analysis software and exported for analysis using Applied Biosystems DataAssist Software.

Statistical Analysis

Data analysis was done with the R programming language. A cutoff for Ct values at 30 was used. MiRNAs with Ct values higher than 30 were considered not detected. Data was normalized using a mean-centering restricted (MCR) strategy. Wylie et al., BMC Res Notes, 4:555 (2011). The MCR method is a modification of the traditional delta Ct method and uses miRNAs which are expressed in all samples for data normalization. The mean of these fully expressed miRNAs in a given sample is subtracted from all miRNAs in that sample.

For the comparison of pre-surgical plasma samples 4 miRNAs (miR-320, -720, -1274b and U6 RNA) were present in all samples and were used for normalization. For the comparison of pre-surgical to post-surgical and post-chemotherapy samples 14 miRNAs (miR-16, miR-19b, miR-24, mirR-146a, mmu-miR-451, U6 rRNA, miR-106a, miR-126-3p, miR-320, miR-191, miR-17, miR-483-5p, miR-1274b, and miR-720) were fully expressed and used for data normalization. After data was normalized, statistical analysis was performed via custom scripts based on the R/Bioconductor package LIMMA (Linear Models for Microarray).

Comparisons between experimental groups were performed using a moderated t-test from LIMMA and p-values were adjusted for multiple testing via the Benjamini-Hochberg method. Fold changes of each miRNA were calculated by the equation 2−ΔΔCt where the comparative cycle threshold (ΔΔCt) is defined as the difference between ΔCt (cancer/experimental) minus ΔCt (control) as previously described. Schmittgen T D, Livak K J, Nat Protoc 3:1101-1108 (2008). A Benjamini-Hochberg adjusted p-value<0.05 was considered statistically significant in comparisons between different groups for each miRNA. As an additional stringency requirement, miRNAs were further considered only if they had at least a 2 fold difference in expression and were present in at least 75% of the samples unless otherwise specified. It is noteworthy that no miRNAs that were specifically expressed in one group but not another were found. Area under the curve (AUC) analysis was performed with the R/Bioconductor package ROC.

### Results

Plasma miRNA Expression Levels in Ovarian Cancer and Cancer Free Subjects

Using plasma isolated miRNAs, comprehensive miRNA expression profiles were generated on presurgical plasma samples from 42 women with confirmed serous epithelial ovarian cancer, 36 women diagnosed with a benign neoplasm, and 23 healthy women with no known pelvic mass (Table 2).

TABLE 2
Subject summary

| | Healthy Controls | Benign Mass | Ovarian Cancer |
|---|---|---|---|
| Number of subjects | 23 | 36 | 42 |
| Age at enrollment | | | |
| (years) | | | |
| mean | 36 | 52 | 64 |
| range | 18-61 | 34-76 | 31-87 |
| Ovarian Cancer Stage | n/a | n/a | |
| IIA | | | 3 |
| IIC | | | 3 |
| IIIA | | | 1 |
| IIIB | | | 12 |
| IIIC | | | 21 |
| IV | | | 2 |

After removing miRNAs that were not present in at least 75% of the samples in each group and those that did not demonstrate at least a 2-fold change in expression (compared to the normalization miRNAs), the remaining miRNAs were analyzed. The 22 most differentially expressed miRNAs between control plasma and pre-surgical plasma from confirmed ovarian cancer subjects are shown in Table 3. The majority of miRNAs (19/22) were underexpressed in plasma from cancer patients compared with controls. In plasma samples from women without a known pelvic mass (healthy controls) compared to presurgical plasma samples from women diagnosed with ovarian cancer, miR-106a, miR-126-3p, miR-146a, miR-150, miR-16, miR-17, miR-19b, miR-20a, miR-223, miR-24 and miR-92a had at least a 10 fold elevation in expression, while miR-106b, miR-191, miR-193a-5p, miR-30b, miR-30a-5p, miR-30c, miR-320, miR-328 were significantly elevated but to a lesser degree (adj p<0.05; AUC>0.8). Three miRNAs, miR-1274a, miR-625-3p and miR-720 were elevated only in cancer samples (adj. p<0.05) however they were not useful discriminators between control and cancer samples (AUC<0.8).

TABLE 3
Comparison of plasma miRNA levels between subject groups

| | control vs | control vs | benign vs |
|---|---|---|---|
| MiRNA | benign | cancer | cancer |
| miR-106a | 4.1 | 10.23 | NS |
| miR-106b | 2.2 | 4.82 | 2.21 |
| miR-126-3p | 4.2 | 16.68 | 3.96 |
| miR-1274a | NS | −2.06 | NS |
| miR-139-5p | 2.4 | NS | NS |
| miR-142-3p | 5.2 | NS | NS |
| miR-146a | 6.3 | 12.79 | NS |
| miR-150 | 8.6 | 20.95 | 2.45 |
| miR-16 | 12.3 | 21.32 | NS |
| miR-17 | 3.7 | 11.14 | 3.03 |
| miR-191 | 6.1 | 7.18 | NS |
| miR-193a-5p | 4.6 | 6.03 | NS |
| miR-19b | 8.2 | 30.36 | NS |
| miR-20a | 4.7 | 18.94 | 4.04 |
| miR-223 | 17.6 | 30.14 | NS |
| miR-24 | 8.7 | 17 | NS |
| miR-30a-5p | 3.9 | 5.86 | NS |
| miR-30b | NS | 8.63 | NS |
| miR-30c | NS | 4.97 | NS |
| miR-320 | 2.5 | 4.03 | NS |
| miR-328 | 4.8 | 7.03 | NS |
| miR-484 | 6.7 | NS | NS |
| miR-486 | 5.2 | NS | NS |
| miR-625-3p | NS | −3.94 | NS |
| miR-660 | 3.7 | NS | NS |
| miR-720 | NS | −2.72 | NS |
| miR-92a | 2.4 | 9.94 | 4.12 |

Comparison of plasma miRNA levels in cancer vs noncancer groups. Comparisons of plasma miRNA levels from subjects diagnosed with ovarian cancer, a benign mass or healthy controls. Only miRNAs that had at least a 2 fold change in expression, were present in at least 75% of each group were used in the analysis. p<0.05 (cancer subjects n=42, benign subjects n=36, healthy controls n=23). NS, not significant.

There were only 6 miRNAs (miR-106b, miR-126, miR-150, miR-17, miR-20a and miR-92a, adj p<0.05) that distinguished benign presurgical plasma from that of cancer subjects suggesting that benign neoplasms are associated with a circulating miRNA profile with some similarity to ovarian cancer (Table 3). Interestingly, three of these differentially expressed miRNAs miR-106b, miR-150 and miR126—have been previously found to be decreased in ovarian cancer tissue. MiR-17, miR-20a and miR-92a belong to the miR-17-92 cluster found on chromosome 13q31.3 and the over expression of this cluster has been associated with several solid cancer types. Concepcion et al., Cancer J. 18:262-267 (2012). Additionally there were 161 miRNAs that were well expressed (ΔCt>3) but were not present in at least 75% of the benign and ovarian cancer subjects.

Differences in miRNA expression between presurgical benign samples and normal controls compared to those observed between controls and ovarian cancer subjects showed differential expression of 17 overlapping miRNAs (Table 3). There are 10 miRNAs that distinguish plasma of healthy control subjects from pre-surgical plasma samples from women with ovarian cancer or a benign neoplasm. In the comparison of healthy controls to cancer plasma samples there are 5 miRNAs, miR-1274a, miR-30b, miR-30c, miR-625-3p and miR-720 that are differentially expressed. MiR-139-5p, miR-142-3p, miR-484, miR-486 and miR-660 are higher in healthy controls when analyzed against benign plasma samples.

Presurgical Plasma miRNAs Associated with Long Term Outcome in Patients with Ovarian Cancer

Pre-surgical plasma miRNA profiles of women who turned out to have ovarian cancer, and for whom there was outcome data (N=26), were compared. Significant differences in miRs-720, miR-20a, miR-223, miR-126-3p and miR-1290 expression were found in women who had short overall survival (SOS) (<2 years, N=19) when compared to women with long overall survival (LOS) (>4 years, N=7) (Table 4).

TABLE 4
Comparison of miRNAs in presurgical
plasma of LOS vs SOS subjects

| | | | Fold | |
|---|---|---|---|---|
| ID | P. Value | adj. P. Val | Change | AUC |
| miR-720 | 0.002 | 0.079 | −4.57 | 0.13 |
| miR-20a | 0.03 | 0.3 | −2.63 | 0.15 |
| miR-223 | 0.03 | 0.3 | 4.474 | 0.73 |
| miR-126-3p | 0.016 | 0.24 | 4.818 | 0.74 |
| miR-1290 | 0 | 0.004 | 14.566 | 0.87 |

MiR-720 and miR-20a were elevated in women who died of their disease less than 2 years after diagnosis. Women who survived >4 years after diagnosis have elevated levels of miR-223, miR-126-3p and miR-1290 in their presurgical plasma (Table 4). However, after correction for multiple testing, only the level of miR-1290 was significantly different between the two groups (adj p value<0.05, AUC of 0.87), Age and clinical stage at diagnosis were comparable for both groups analyzed.

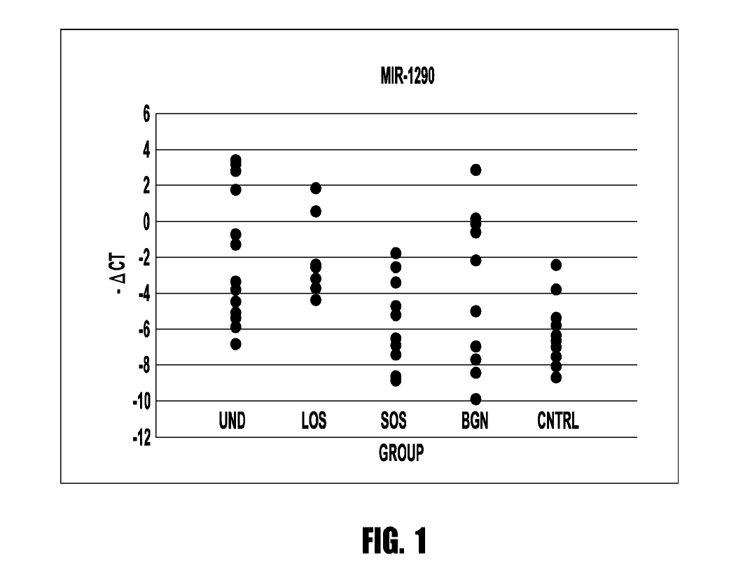

The expression levels of miR-1290 detected in all samples included in this study are shown in FIG. 1. MiR-1290 was present in all of the samples from LOS subjects (7/7) tested but was detected in only 12/19 SOS, 15/16 UND, 13/23 control and 12/36 benign subjects. In this dataset, miR-1290 was calculated to have a sensitivity of 63.3% and 100% specificity for long term survival in subjects with ovarian cancer (e.g. considering only people with ovarian cancer) with a PPV and PNV of 100% and 75%, respectively. Seventy-one miRNAs were found to be significantly expressed (ΔCT≧3) in at least 2 individuals of either the LOS or SOS subject group although further analysis could not be performed.

Changes of miRNA Expression in Plasma of Ovarian Cancer Patients after Surgical Treatment and Chemotherapy.

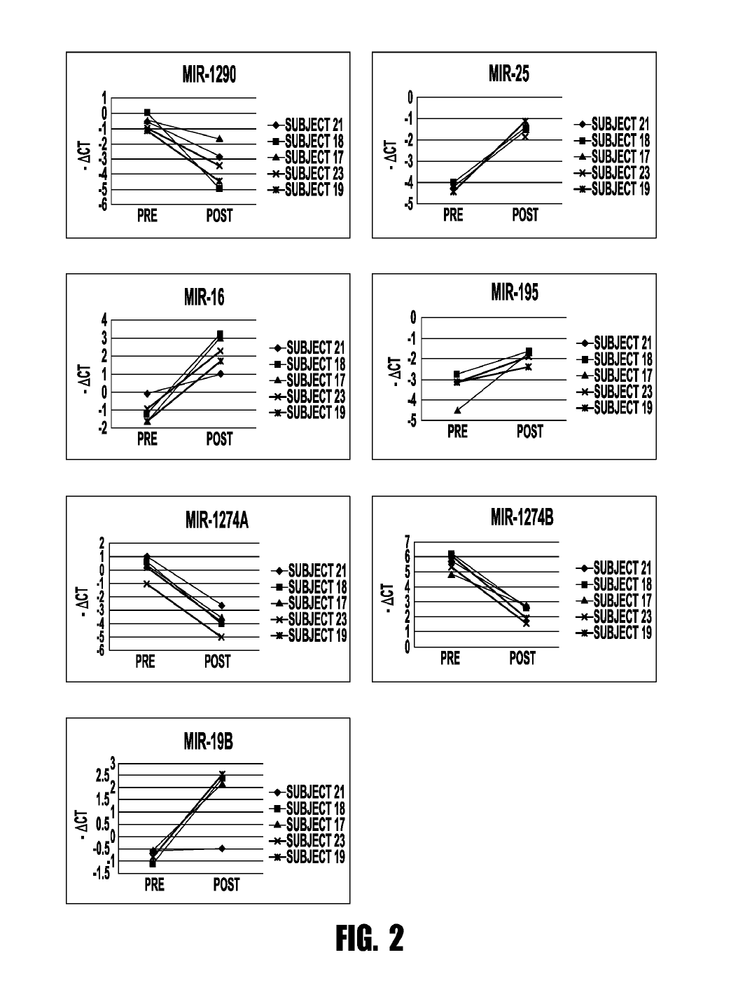

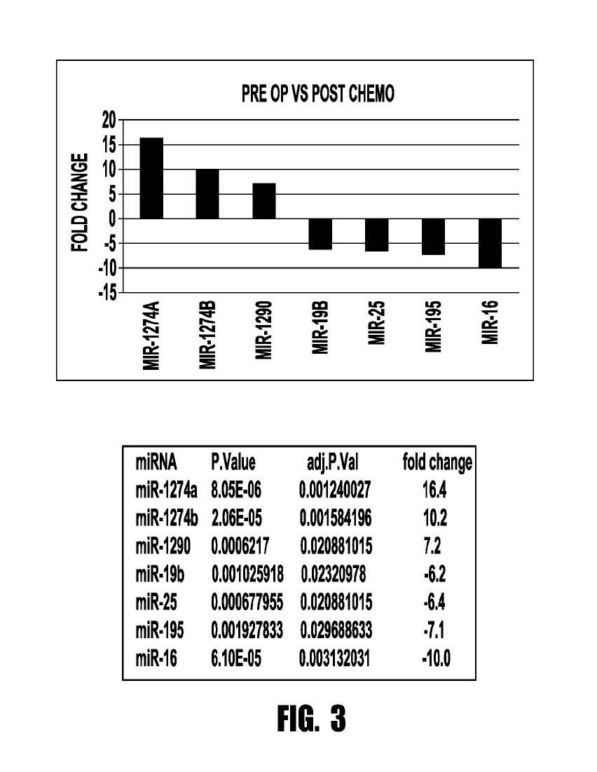

Five LOS subjects had presurgical and post chemotherapy plasma samples available for analysis. There were 7 miRNAs which were differentially expressed between matched presurgical and post chemotherapy samples. These changes are summarized in FIGS. 2 and 3. MiR-1274a, miR-1274b and miR-1290 displayed consistent decreases in plasma levels after treatment, whereas miR-19b, -25, -195 and -16 were more highly expressed in postchemotherapy samples when compared to their respective presurgical plasma sample (adj p<0.05). Relevant to current treatment of ovarian cancer, the expression of miR-16 is 10 fold higher in post chemotherapy plasma samples compared to the presurgical sample.

The comparison of presurgical to immediate postsurgical plasma samples, measured before chemotherapy, did not show any significant differences in miRNA expression in paired analyses from the same subjects (n=9; data not shown).

### Discussion

In order to address several unanswered questions regarding the utility of circulating miRNAs as biomarkers in ovarian cancer, blood samples were analyzed from women diagnosed with ovarian cancer, benign neoplasm or healthy control samples. A number of considerations were taken into account prior to analyzing miRNAs from plasma. For example, the use of circulating miRNAs as biomarkers of cancer have come under increased scrutiny, due to lack of standardized miRNA isolation methods and the possibility of contamination of RBC, lymphoid and myeloid derived miRNAs. Pritchard et al. reported the potential for confounding factors related to miRNA contamination from lysed cells in previously reported studies including ovarian cancer. Pritchard et al., Cancer Prev Res 5:492-497 (2012). Therefore, in order to minimize the contribution of miRNAs from lysed PBMCs, RBCs, platelets and other cellular debris, plasma was isolated within 5 hours of blood draw. Secondly, after an initial low speed centrifugation, the plasma was removed and ultracentrifuged leaving only protein bound miRNA in the supernatant. It has been suggested that the majority of disease related miRNAs found in circulation are vesicle independent originating from the lysis of affected cells. The inventors confirmed the absence cellular debris including microvessicles and exosomes in the supernatant by electron microscopy. The stable presence of microvessicle and exosome-free miRNAs in circulation is dependent upon being bound to Ago 2 in order to protect them from degradation. Arroyo et al., Proc Natl Acad Sci USA 108:5003-5008 (2011). Previous studies have identified circulating miRNAs in serum (Chung et al., Int J Gynecol Cancer 23:673-679 (2013)), exosomes (Taylor D D, Gercel-Taylor C, Gynecol Oncol 110:13-21 (2008)) and whole blood (Häusler et al, Br J Cancer 103:693-700 (2010)) from ovarian cancer subjects. Resnick et al. identified 8 miRNAs in the serum of 28 ovarian cancer patients. Resnick et al., Gynecol Oncol 112:55-59 (2009). Mir-21, -92, -93, -126 and -29a were elevated in presurgical serum of cancer patients while miR-155, -127 and -99b were lower in cancer patients compared to healthy controls. More recently, Chung et al. (2013) found miR-132, -26a -145 and let7b to be underexpressed in serum of ovarian cancer patients. In a study by Taylor and Gercel-Taylor (2008), it was found that exosomes isolated from serum of ovarian cancer expressed higher levels of miR-21, -141, -200a, 200b, 200c, 203, 205 and -214 when compared to patients with benign ovarian disease. Häusler et al. (2010) measured miRNA levels in 24 patients with relapsed ovarian cancer. In comparison to controls, they found 4 miRNAs that remained significantly expressed after adjustment for multiple testing; miR-30c1-3p, 342-3p, 181a-3p and 450b-5p. A comparison of circulating miRNAs identified to distinguish control and/or benign from cancer subjects from previous studies failed to identify common miRNAs expressed in the same subject group. Interestingly Resnick et al. (2009) found miR-126 to be elevated in serum from cancer patients while more robust levels of miR-126 were found in plasma samples from healthy control and benign mass subjects. This discrepancy among ours and previous studies may be attributable to the source of miRNAs (whole blood, plasma, serum or exosomes) from primary ovarian cancer or relapse patients, and/or the platform (qPCR or microarray based) used for analysis. While the inventors recognize that some highly expressed miRNAs relevant to ovarian cancer may go undetected due to degradation, tissue sequestration or microvessicle/exosome containment, those that they have identified may be seminal to detection and outcome of the disease. This is the first serous epithelial ovarian cancer study to specifically focus on circulating, protein bound miRNAs in plasma, free of cellular debris, microvessicles and exosomes.

Initially, the inventors set out to determine whether specific circulating miRNAs could be used to detect the presence of ovarian cancer in subjects being evaluated for a pelvic mass. The expression patterns of 754 miRNAs in pre-surgical plasma samples from 42 women with confirmed serous epithelial ovarian cancer compared to 36 plasma samples collected from women who turned out to have a benign pelvic mass at surgery were assessed. Both of these groups were also compared to age matched healthy subjects without evidence of pelvic pathology. Interestingly, both cancerous and benign neoplasms were generally associated with lower levels of a group of 22 circulating miRNAs, as shown in Table 3. A much smaller subset of 6 miRNAs were significantly different between groups of women with cancer vs. a benign neoplasm. Of these, miR-106b, miR-150 and miR-126 have been previously reported to have decreased expression in ovarian cancer tissue compared to noncancerous ovarian tissue. There does not seem to be an obvious overlap in gene targets/pathways of these 3 miRNAs to explain their lowered expression in ovarian cancer. Significantly elevated expression of miR-17, miR-20a and miR-92a of the miR-17-92 cluster was found in pre-surgical plasma from women with a benign neoplasm compared to women with ovarian cancer. The role and the function of these miRNAs in cancer is difficult to identify since their function is both cell type and context specific. Olive et al., Int J Biochem Cell Biol. 42:1348-1354 (2010).

In 2011, the TCGA published and made available genetic, epigenetic and expression data on 489 high grade serous ovarian cancer tissue samples. The inventors downloaded and analyzed level 3 miRNA microarray expression data of women with ovarian who had not been previously treated for their disease (i.e. adjuvant, chemotherapy). The 6 miRNAs (miR-106b, miR-126, miR-150, miR-17, miR-20a and miR-92a), that were found to distinguish presurgical plasma from benign or cancer patients were well expressed in ovarian cancer tissue from this dataset; however, without comparable miRNA expression data from samples with benign ovarian disease the significance of miRNA expression of miRNAs in ovarian cancer tissue could not be determined.

To take into account the possibility that highly expressed miRNAs in a small number of subjects could also distinguish benign from ovarian cancer subjects, the miRNA inclusion criteria were relaxed to include miRNAs that had a ΔCt>3 and were present in at least 2 subjects of either the benign or ovarian cancer group. One hundred sixty one samples met this modified criteria, of these 21 were present in presurgical plasma from subjects with a benign mass and 22 were present in presurgical plasma from women subsequently diagnoses with ovarian cancer. The relevance of these uniquely expressed miRNAs in either benign or ovarian cancer presurgical plasma will need to be evaluated in a larger dataset.

The utility of presurgical circulating plasma miRNA in predicting outcome of those subjects who turned out to have ovarian cancer at operation was also examined. Using follow up data available on 26 ovarian cancer subjects in the study, pre-surgical plasma miRNA profiles of women who died within 2 years of diagnosis (but survived at least 6 months post-surgery) were compared with those who survived 4 years or more. Although 5 year survival is standard, the inventors chose to include subjects with at least 4 year survival for the LOS group to include enough subjects for meaningful analysis. Distinct differences were found between these two groups. There were 5 miRNAs (miR-720, miR-20a, miR-223, miR-126-3p and miR-1290) that were differentially expressed between LOS and SOS subjects, however only miR-1290 remained significant after multiple testing adjustments (adj p=0.004) Table 3.

There were an additional 71 miRNAs that were significantly present (ΔCt≧3) but were detectable in less than 50% of the LOS or SOS group. Nineteen of these miRNAs were only found in presurgical plasma samples from SOS while 4 were present only in LOS subjects. Due to the small number of subjects further analysis could not be performed to determine significance. Using the criteria for LOS and SOS, TCGA miRNA microarray level 3 data were analyzed for corresponding tissue miRNA associated with outcome. None of the miRNAs available for analysis were significant.

The expression of miR-1290 may directly influence outcome in women with ovarian cancer and its expression is independent of age and cancer stage at diagnosis. One highly scored potential gene target of miR-1290 is epithelial membrane protein 2 (EMP2) (Table 5). EMP2 is a tetraspan protein that facilitates transmembrane activity of specific integrins and can act as an oncogene in a number of cancers (Habeeb et al., Cancer. 1164718-4726 (2010)) and its elevated expression has been associated with poor outcome in patients with endometrial cancer. Wadehra et al., Cancer. 107:90-98 (2006). EMP2 is also expressed in ovarian cancer although unlike with endometrial cancer, the level of expression was not associated with patient survival. In vitro and in vivo treatment of ovarian cancer cells with an engineered bivalent antibody against EMP2 inhibited cell growth and resulted in cell death. Fu et al., Clin Cancer Res. 16:3954-3963 (2010). It is conceivable that elevated expression of miR-1290 observed in the LOS subjects led to decreased expression of EMP2 and consequently resulted/contributed to their long term cancer free status. Finally, a comparison of presurgical and post surgical plasma samples from matched individuals did not show any significant differences. This is most likely explained by the timing of ascertainment which was approximately 2 weeks post surgery and may have been too soon to detect relevant changes. However, analysis of presurgical and post chemotherapy plasma (cured) samples from LOS subjects showed a dramatic change in miRNA profiles. Although based on a relatively small number of matched pre-surgical and post chemotherapy sample pairs (5), these miRNAs (miR-1274a, miR-1274b, miR-1290, miR-19b, miR-25, miR-195, and miR-16) maintained their significance even after multiple testing corrections (adj p<0.05). Interestingly, miR-1290 whose elevated expression was predictive of survival in presurgical plasma is decreased in long overall survivors (>4 years) of ovarian cancer suggesting that it may be induced/regulated in the presence of ovarian cancer. Although suppressed in pre-surgical plasma miR-19b, miR-25, miR-195 and miR-16 are elevated in the LOS subjects. In particular there is a 10 fold elevation of miR-16 in LOS post chemotherapy plasma samples when compared to presurgical plasma samples. A recent paper by Pouliot et al. showed that transfection of a miR-16 mimic into cisplatin-resistant cells resulted in a 5 fold increase in cisplatin sensitivity although the mechanism is undefined. Pouliot et al., Cancer Res. 72:5945-5955 (2012). Cisplatin is a common chemotherapy used to treat ovarian cancer among others and one could envision that chemosensitivity maintained by elevated levels of miR-16 during the chemotherapy contributes to a favorable outcome in the LOS subjects. To confirm effects of miR-1290 and miR-16 to support long term cancer free status in ovarian cancer, confirmation and validation in a larger multi-site cohort will be needed. Given the general nature of miRNAs associated with cancers, it is highly likely these miRNAs play a pivotal role in cell proliferation and/or metastasis but their specific role in affecting ovarian cancer outcome, whether directly or indirectly, remains to be determined. Further studies will be needed to confirm the extent that circulating levels miRNAs of are reflective of disease status.

TABLE 5
Top 10 gene targets of miR-1290

| Target | Target | Gene | |
|---|---|---|---|
| Rank | Score | Symbol | Gene Description |
| 1 | 100 | RASGRF2 | Ras protein-specific guanine |
| | | | nucleotide-releasing factor 2 |
| 2 | 98 | EMP2 | epithelial membrane protein 2 |
| 3 | 98 | CCDC142 | coiled-coil domain containing 142 |
| 4 | 97 | KLHL6 | kelch-like 6 (Drosophila) |
| 5 | 97 | MAF | v-maf musculoaponeurotic fibrosarcoma |
| | | | oncogene homolog (avian) |
| 6 | 97 | KLHL5 | kelch-like 5 (Drosophila) |
| 7 | 97 | ONECUT2 | one cut homeobox 2 |
| 8 | 97 | PRKAA2 | protein kinase, AMP-activated, |
| | | | alpha 2 catalytic subunit |
| 9 | 96 | OSBPL6 | oxysterol binding protein-like 6 |
| 10 | 95 | KDM5A | lysine (K)-specific demethylase 5A |

### Example 2

### Analysis of miRNA Expression in Additional Pre-Op Plasma Samples

Since completion of the work described in Example 1, additional pre-op plasma samples were analyzed for miRNA expression. These included 15 benign and 26 ovarian cancer samples. These samples were analyzed using the same procedures described in Example 1. Alone, the sample size used in this additional work was too small to achieve statistical significance. However, when these samples were combined with the data described in Example 1, some previously identified miRNAs were found to retain their significance, and more importantly new miRNAs which could be used to diagnose ovarian cancer identified. In particular, miR-22-p5, miR-483-5p, and miR-1274b were found to be useful. The new results were combined with the previous data to generate Tables 6-8, which are provided below.

TABLE 6
Analysis of Benign vs. Cancer Samples
Combined data sets
•Benign vs cancer

| | ID | FC | NrSamp1 | AllSamp1 | NrSamp2 | AllSamp2 |
|---|---|---|---|---|---|---|
| ** | miR-92a | 2.40913 | 51 | 51 | 67 | 68 |
| ** | miR-150 | 2.593877 | 49 | 51 | 55 | 68 |
| ** | miR-126 | 2.598671 | 45 | 51 | 57 | 68 |
| ## | miR-122-5p | 2.879237 | 44 | 51 | 47 | 68 |
| ** | miR-20a | 2.989274 | 43 | 51 | 59 | 68 |

** identified in original dataset: significant in combined dataset
## new-identified in combined dataset

TABLE 7
Analysis of Control vs. Cancer Samples
Combined data sets
•Control vs cancer

| | ID2 | FC | InvFC | NrSamp1 | AllSamp1 | NrSamp2 | AllSamp2 |
|---|---|---|---|---|---|---|---|
| ** | miR-150 | 14.74716 | 0.06781 | 23 | 23 | 55 | 68 |
| ** | miR-223 | 11.7989 | 0.084754 | 23 | 23 | 62 | 68 |
| ** | miR-126 | 7.070025 | 0.141442 | 23 | 23 | 57 | 68 |
| ** | miR-24 | 6.665412 | 0.150028 | 23 | 23 | 62 | 68 |
| ** | miR-16 | 6.59511 | 0.151627 | 23 | 23 | 56 | 68 |
| ** | miR-19b | 5.741124 | 0.174182 | 23 | 23 | 60 | 68 |
| ** | miR-146a | 5.688756 | 0.175785 | 23 | 23 | 59 | 68 |
| ** | miR-20a | 5.49354 | 0.182032 | 22 | 23 | 59 | 68 |
| ** | miR-328 | 4.011522 | 0.249282 | 20 | 23 | 55 | 68 |
| ** | miR-191 | 3.360244 | 0.297597 | 23 | 23 | 55 | 68 |
| ** | miR-17 | 3.169985 | 0.315459 | 23 | 23 | 57 | 68 |
| ** | miR-106a | 3.023458 | 0.330747 | 23 | 23 | 54 | 68 |
| ** | miR-30a-5p | 3.005132 | 0.332764 | 22 | 23 | 54 | 68 |
| ** | miR-193a-5p | 2.992808 | 0.334134 | 21 | 23 | 57 | 68 |
| ** | miR-92a | 2.611134 | 0.382975 | 23 | 23 | 67 | 68 |
| ** | miR-320 | 1.888886 | 0.529413 | 23 | 23 | 68 | 68 |
| ## | miR-483-5p | 0.362936 | 2.755308 | 23 | 23 | 63 | 68 |
| ## | miR-1274b | 0.252036 | 3.967689 | 23 | 23 | 66 | 68 |
| ** | miR-1274a | 0.163584 | 6.113067 | 23 | 23 | 60 | 68 |
| ** | miR-720 | 0.135564 | 7.376588 | 23 | 23 | 66 | 68 |

** identified in original dataset: significant in combined dataset
## new-identified in combined dataset

TABLE 8
Analysis of Control vs. Benign Samples
Combined data sets
•control vs benign

| | ID2 | FC | InvFC | NrSamp1 | AllSamp1 | NrSamp2 | AllSamp2 |
|---|---|---|---|---|---|---|---|
| ** | miR-126 | 2.720631 | 0.367562 | 23 | 23 | 45 | 51 |
| ** | miR-1274a | 0.263317 | 3.797697 | 23 | 23 | 48 | 51 |
| ** | miR-1274b | 0.258381 | 3.870261 | 23 | 23 | 51 | 51 |
| ## | miR-130a | 2.258152 | 0.44284 | 21 | 23 | 39 | 51 |
| ** | miR-142-3p | 4.032477 | 0.247987 | 21 | 23 | 39 | 51 |
| ** | miR-146a | 3.509389 | 0.28495 | 23 | 23 | 48 | 51 |
| ## | miR-146b | 3.28085 | 0.304799 | 23 | 23 | 27 | 51 |
| ** | miR-150 | 5.685373 | 0.17589 | 23 | 23 | 49 | 51 |
| ## | miR-15b | 4.108139 | 0.243419 | 17 | 23 | 18 | 51 |
| ** | miR-16 | 3.983666 | 0.251025 | 23 | 23 | 46 | 51 |
| ** | miR-191 | 2.989276 | 0.334529 | 23 | 23 | 44 | 51 |
| ** | miR-193a-5p | 2.542165 | 0.393366 | 21 | 23 | 44 | 51 |
| ## | miR-197 | 3.334074 | 0.299933 | 19 | 23 | 7 | 51 |
| ## | miR-206 | 0.108809 | 9.190436 | 6 | 23 | 12 | 51 |
| ## | miR-222 | 3.012576 | 0.331942 | 23 | 23 | 22 | 51 |
| ** | miR-223 | 8.42117 | 0.118748 | 23 | 23 | 46 | 51 |
| ** | miR-24 | 4.311441 | 0.231941 | 23 | 23 | 47 | 51 |
| ## | miR-30e-3p | 2.972178 | 0.336454 | 17 | 23 | 20 | 51 |
| ## | miR-320b | 3.046257 | 0.328272 | 19 | 23 | 30 | 51 |
| ** | miR-328 | 3.279759 | 0.3049 | 20 | 23 | 40 | 51 |
| ## | miR-335 | 2.351306 | 0.425296 | 17 | 23 | 27 | 51 |
| ## | miR-342-3p | 7.332416 | 0.136381 | 22 | 23 | 22 | 51 |
| ## | miR-423-5p | 2.266413 | 0.441226 | 20 | 23 | 35 | 51 |
| ** | miR-484 | 3.059705 | 0.326829 | 22 | 23 | 46 | 51 |
| ** | miR-486 | 2.339409 | 0.427458 | 22 | 23 | 32 | 51 |
| ## | miR-502-3p | 9.33411 | 0.107134 | 5 | 23 | 4 | 51 |
| ## | miR-520c-3p | 0.026002 | 38.45818 | 2 | 23 | 8 | 51 |
| ## | miR-636 | 0.24713 | 4.046453 | 8 | 23 | 11 | 51 |
| ** | miR-720 | 0.153575 | 6.511481 | 23 | 23 | 51 | 51 |
| ## | miR-99b* | 0.090815 | 11.01145 | 9 | 23 | 16 | 51 |

** identified in original dataset: significant in combined dataset
## new-identified in combined dataset

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of diagnosing ovarian cancer in a subject, comprising
identifying the expression level of at least one diagnostic miRNA selected from the group consisting of miR-106a, miR-106b, miR-126, miR-1274a, miR-139-5p, miR-142-3p, miR-146a, miR-150, miR-16, miR-17, miR-191, miR-193a-5p, miR-30a-5p, miR-19b, miR-20a, miR-223, miR-24, miR-30b, miR-30c, miR-320, miR-328, miR-484, miR-486, miR-625-3p, miR-660, miR-720, miR-92a, miR-122-5p, miR-483-5p, and miR-127-4b in a biological sample from the subject, comparing the expression level of the at least one diagnostic miRNA to control expression levels, and diagnosing the subject as having or being at an increased risk of having ovarian cancer if the subject has a changed expression level of the one or more diagnostic miRNA.

2. The method of claim 1, wherein the changed level is an increased level, and wherein the diagnostic miRNA is selected from the group consisting of miR-1274a, miR-625-3p, miR-720, miR-483-5p, and miR-1274b.

3. The method of claim 1, wherein the changed level is a decreased level, and wherein the diagnostic miRNA is selected from the group consisting of miR-106a, miR-106b, miR-126-3p, miR-139-5p, miR-142-3p, miR-146a, miR-150, miR-16, miR-17, miR-191, miR-193a-5p, miR-19b, miR-20a, miR-223, miR-24, miR-30b, miR-30c, miR-320, miR-328, miR-484, miR-486, miR-660, miR-92a, and miR-122-5p.

4. The method of claim 3, wherein the diagnostic miRNA is selected from the group consisting of miR-106a, miR-126-3p, miR-146a, miR-150, miR-16, miR-17, miR-19b, miR-20a, miR-223, miR-24, and miR-92a.

5. The method of claim 1, wherein the subject is human.

6. The method of claim 1, wherein the ovarian cancer is serous epithelial ovarian cancer.

7. The method of claim 1, wherein the expression level of a plurality of diagnostic miRNAs are identified.

8. The method of claim 1, wherein the biological sample is selected from the group consisting of blood, plasma, and serum.

9. The method of claim 1, wherein the biological sample is plasma.

10. The method of claim 9, wherein the plasma is ultracentrifuged before determining the level of diagnostic miRNA in the plasma.

11. The method of claim 1, wherein the level of diagnostic miRNA is determined using a polymerase chain reaction amplification-based assay.

12. The method of claim 1, further comprising the step of administering or prescribing a therapeutic agent targeted to ovarian cancer to a subject diagnosed as having ovarian cancer.

13. A method of providing a prognosis for a subject having ovarian cancer, comprising
identifying the expression level of at least one prognostic miRNA selected from the group consisting of miR-720, miR20a, miR-223, miR-126-3p, and miR-1290 in a biological sample from the subject, comparing the expression level of the at least one prognostic miRNA to control expression levels, and identifying the subject as having a poor prognosis if the subject has a changed expression level of the one or more prognostic miRNA.

14. The method of claim 13, wherein the prognostic miRNA is miR-1290.

15. The method of claim 13, wherein the subject is human.

16. The method of claim 13, wherein the ovarian cancer is serous epithelial ovarian cancer.

17. The method of claim 13, wherein the expression level of a plurality of prognostic miRNAs are identified.

18. The method of claim 13, wherein the biological sample is selected from the group consisting of blood, plasma, and serum.

19. The method of claim 13, wherein the biological sample is plasma.

20. The method of claim 13, wherein the plasma is ultracentrifuged before determining the level of prognostic miRNA in the plasma.

21. The method of claim 13, wherein the level of prognostic miRNA is determined using a polymerase chain reaction amplification-based assay.

22. A kit for diagnosing ovarian cancer in a subject, comprising:
one or more detector means specific for at least one diagnostic miRNA selected from the group consisting of miR-106a, miR-106b, miR-126-3p, miR-1274a, miR-139-5p, miR-142-3p, miR-146a, miR-150, miR-16, miR-17, miR-191, miR-193a-5p, miR-19b, miR-20a, miR-223, miR-24, miR-30b, miR-30c, miR-320, miR-328, miR-484, miR-486, miR-625-3p, miR-660, miR-720, miR-92a, miR-122-5p, miR-483-5p, and miR-127-4b
means for comparing the expression level of the at least one diagnostic miRNA to the level of a corresponding control, and
instructions for using the kit to diagnose a subject as having or being at an increased risk of having ovarian cancer when the expression level of the at least one diagnostic miRNA is changed compared to control expression levels.

21. The kit of claim 22, wherein the changed level is an increased level, and wherein the diagnostic miRNA is selected from the group consisting of miR-1274a, miR-625-3p, miR-720, miR-483-5p, and miR-1274b.

22. The kit of claim 22, wherein the changed level is a decrease level, and wherein the diagnostic miRNA is selected from the group consisting of miR-106a, miR-106b, miR-126-3p, miR-139-5p, miR-142-3p, miR-146a, miR-150, miR-16, miR-17, miR-191, miR-193a-5p, miR-19b, miR-20a, miR-223, miR-24, miR-30b, miR-30c, miR-320, miR-328, miR-484, miR-486, miR-660, miR-92a, and miR-122-5p.

23. The kit of claim 22, wherein the kit includes a plurality of different detector means suitable for determining the expression level of a plurality of diagnostic miRNA.
